(19) United States
(12) Patent Application Publication
(10) Pub. No.: US 2012/0029639 A1
(43) Pub. Date: Feb. 2, 2012

(54) INTERBODY SPINAL IMPLANTS AND INSERTION TECHNIQUES

(75) Inventors: Jonthan E. Blackwell, Arlington, TN (US); Anthony J. Melkent, Memphis, TN (US); Kidong Yu, Memphis, TN (US)

(73) Assignee: WARSAW ORTHOPEDIC, INC., Warsaw, IN (US)

(21) Appl. No.: 12/845,809

(22) Filed: Jul. 29, 2010

(51) Int. Cl. A61F 2/44

(52) U.S. Cl. 623/17.16

(57) ABSTRACT

An implant for a spinal column is disclosed that is capable of being inserted into a patient in a first orientation having a reduced or minimum height and then rotated to a second orientation providing a second or maximum height, wherein in each orientation the height of the implant extends in a direction from one endplate toward the other endplate of adjacent vertebrae. The implant includes first and second side-by-side elongate members that are separately rotated from the first orientation to the second orientation after implantation. The first and second implant members are interconnected by a housing that maintains the first and second members in side-by-side relation.

### BACKGROUND

The present invention relates generally to interbody spinal implants and to methods and systems for inserting one or more interbody spinal implants between adjacent vertebrae.

Several techniques and systems have been developed for correcting and stabilizing the spine and for facilitating fusion at various levels of the spine. The spinal anatomy including the bony structure of vertebral bodies, vascular structures, neural structures, musculature, and other vital tissue along the spinal column make it difficult to position an interbody implant in the disc space between adjacent vertebral bodies. In addition, when an implant is placed into a disc space, the channel or path that the implant took to enter the disc space provides a path for retrograde movement of the implant from the disc space. Also, when approaches other than a direct anterior approach are taken, current implants do not provide the desired fit and angulation between endplates of the adjacent vertebrae.

Preservation of cortical bone of the endplates is desired in order to maximize the stability of the fusion area. Therefore, the cortical bone of the endplates of the superior and inferior vertebrae is ideally left entirely or substantially intact. Positioning some implants from approaches that parallel or extend substantially parallel to the sagittal plane can be difficult to achieve with an interbody implant. With some implants, surface area contact between the implant and the hard cortical bone of the endplate can be too small so that the implant subsides too much and tends to want to break through the endplates. Unilateral fixation is not always an option because of stability issues of a narrow implant. While a lateral approach to the disc space avoids certain critical anatomical structures that impede access in other approaches, insertion of implants into the disc space from a lateral approach is challenging without performing a partial removal of the endplate and osteophytes. As a result, additional improvements in spinal fusion implants and insertion instruments and techniques are needed that make utilization of a lateral approach more palatable, although utilization of such implants and instrument is not necessarily limited to a lateral approach.

### SUMMARY

According to one aspect, an implant for a spinal column is disclosed that is capable of being inserted into a patient in a first orientation having a reduced or minimum height and then rotated to a second orientation providing a second greater height, wherein in each orientation the height of the implant extends in a direction from one endplate to the other endplate of adjacent vertebrae. In one embodiment, the implant includes first and second side-by-side elongated members that are separately rotated from the first orientation to the second orientation after insertion in the disc space. In another embodiment, the first and second implant members are interconnected by a housing that is located about mid-length of the first and second members.

In yet another aspect, an implant for a spinal column is disclosed that is capable of being inserted into a patient in a first orientation having a reduced or minimum height and then rotated to a second orientation providing a second or maximum height, wherein in each orientation the height of the implant extends in a direction from one endplate to the other endplate of adjacent vertebrae. In one embodiment, the implant includes a first member that is connected with a housing, and the first implant member is rotated from the first orientation to the second orientation while connected to the housing in the disc space. After the first member of the implant is rotated, a second member of the implant is inserted in a first orientation into the disc space and connected to the housing. The second implant member of the implant is then rotated from its first orientation to the second orientation while in side-by-side relation to the first member of the implant.

In a further aspect, an implant for a spinal column is disclosed that includes first and second elongated members extending in side-by-side relation that are rotatable in a housing that interconnects the first and second members. The implant includes a height that tapers from an outer sidewall of the first member to an outer sidewall of the second member when the first and second members are rotated in the housing to an implantation orientation. In one embodiment, the housing is located about mid-length along the first and second members and extends completely around the first and second members so that each of the first and second members includes first and second portions projecting from the housing in opposite directions from one another.

In another aspect, an implant for a spinal column is disclosed that includes at least three components. The components includes an anterior rotating spacer member, a posterior rotating spacer member, and a center housing extending along the sagittal plane that holds the spacer members together. The housing allows both spacer members to rotate around its respective central longitudinal axis so that the spacer members can be inserted into the disc space in a first orientation and then rotated for implantation at a second orientation. In the first orientation the insertion height of the implant is smaller than its implanted height obtained after rotation of the spacer members. In one embodiment, each spacer member is rotated 90 degree around its central longitudinal axis between the first and second orientations.

During implantation of the implant into the disc space in a lateral approach, the implant is maintained so that its insertion height is oriented toward the endplates until the implant is properly positioned along the transverse length of intervertebral space. After the implant is in the desired position in its insertion height orientation, the spacer members are rotated to an implantation orientation where superior and inferior bone engaging surfaces of the spacer members contact the adjacent endplates and distract the vertebrae to restore the intervertebral height. The spacer members can either be rotated independently or simultaneously while the housing preserves both insertion and final widths of the spacing between the spacer members. The housing can also be configured to allow the width of the spacing between the spacer members to be varied. In one embodiment, each of the spacer members includes first and second portions extending in opposite directions from the housing, where the first and second portions each include convexly curved superior and inferior bone engaging surfaces extending from a respective end of the spacer member to the housing. In a further embodiment, the bone engaging surfaces include ridges, teeth or other suitable engagement structure to securely engage the implant to the respective adjacent endplate. In yet a further embodiment, the spacer members include one or more cavities or holes to receive bone growth material and/or bone growth between the adjacent vertebrae.

In another implantation method, the implant is provided with only one spacer member engaged to the housing initially. The housing and first spacer member are implanted in the disc space with the spacer member rotated in a reduced height orientation. The first spacer member is then rotated about its longitudinal axis to a second orientation where its upper and lower bone engaging surface are positioned in contact with the endplates of the adjacent vertebrae. A second spacer member is then positioned in the disc space in a reduced height orientation alongside the first spacer member. The second spacer member is engaged to the housing while in its insertion orientation and then rotated to an implanted orientation adjacent to the first spacer member to contact its upper and lower bone engaging surfaces with the endplates of the adjacent vertebrae. The housing maintaining the spacing between the sides of the first and second spacer members.

Related features, aspects, embodiments, objects and advantages of the present invention will be apparent from the following description.

### BRIEF DESCRIPTION OF THE DRAWINGS

FIG. 1 is a diagrammatic plan view looking toward the axial plane of an endplate of a vertebral body of a spinal column with an interbody spinal implant positioned thereon.

FIG. 2 is a diagrammatic elevation view looking toward the coronal plane at a vertebral level of the spinal column including the vertebral body and interbody spinal implant of FIG. 1.

FIG. 3 is a diagrammatic elevation view looking toward the sagittal plane at a vertebral level of the spinal column including the vertebral body and interbody spinal implant of FIG. 1.

FIG. 4 is a diagrammatic elevation view looking toward the sagittal plane at a vertebral level of the spinal column including the vertebral body and interbody spinal implant of FIG. 1 with the interbody spinal implant positioned in an initial insertion orientation.

FIG. 5 is a perspective rear view of a spinal interbody implant with members thereof in an initial insertion orientation.

FIG. 6 is a plan view of the spinal interbody implant of FIG. 5 in its initial insertion orientation,

FIG. 7 is an end elevation view of the spinal interbody implant of FIG. 5 in its initial insertion orientation with the vertebrae of the vertebral level shown diagrammatically.

FIG. 8 is a side elevation view of the spinal interbody implant of FIG. 5 in its initial insertion orientation with the vertebrae of the vertebral level shown diagrammatically.

FIG. 9 is a perspective rear view of the spinal interbody implant of FIG. 5 with members thereof in an implantation orientation.

FIG. 10 is a plan view of the spinal interbody implant of FIG. 9 in its implantation orientation

FIG. 11 is an end elevation view of the spinal interbody implant of FIG. 9 in its implantation orientation with the vertebrae of the vertebral level shown diagrammatically.

FIG. 12 is a side elevation view of the spinal interbody implant of FIG. 9 in its final implantation orientation with the vertebrae of the vertebral level shown diagrammatically.

FIGS. 13A-D illustrate another embodiment spinal interbody implant and an insertion sequence therefore.

FIG. 14 is a perspective view of another embodiment spinal interbody implant in an insertion orientation.

FIG. 15 is a perspective view of an implant body of the spinal interbody implant of FIG. 14 removed from the housing.

FIG. 16 is a perspective view of another embodiment housing.

FIGS. 17A-17B show another embodiment interbody spinal implant with an implant member in an insertion orientation connected with housings of FIG. 16 at each end of the implant member.

FIGS. 18A-18B show the interbody spinal implant member of FIGS. 17A-17B in an implanted orientation.

FIGS. 19A-19J illustrate an insertion technique of another embodiment interbody spinal implant.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Methods, techniques, instrumentation and implants are provided to restore and/or maintain a collapsed, partially collapsed, damaged, diseased, or otherwise impaired spinal disc space at a desired disc space height and adjacent endplate orientation. The instruments and implants may be used in techniques employing minimally invasive instruments and technology to access the disc space, although access in non-minimally invasive procedures is also contemplated. Access to the collapsed disc space can be uni-portal, bi-portal, or multi-portal, but is preferentially uni-portal. The instruments and implants may also be employed in a direct lateral approach to the spinal disc space, although other approaches are also contemplated, including antero-lateral, postero-lateral, oblique, posterior, and anterior approaches. Also, the surgical methods, techniques, instruments and implants may find application at all vertebral segments of the spine, including the lumbar, thoracic and cervical spinal regions.

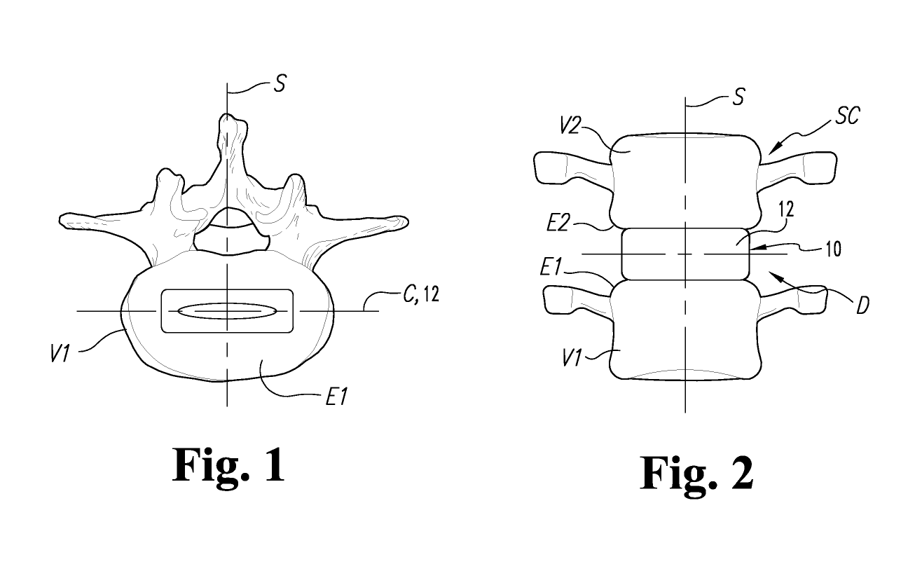

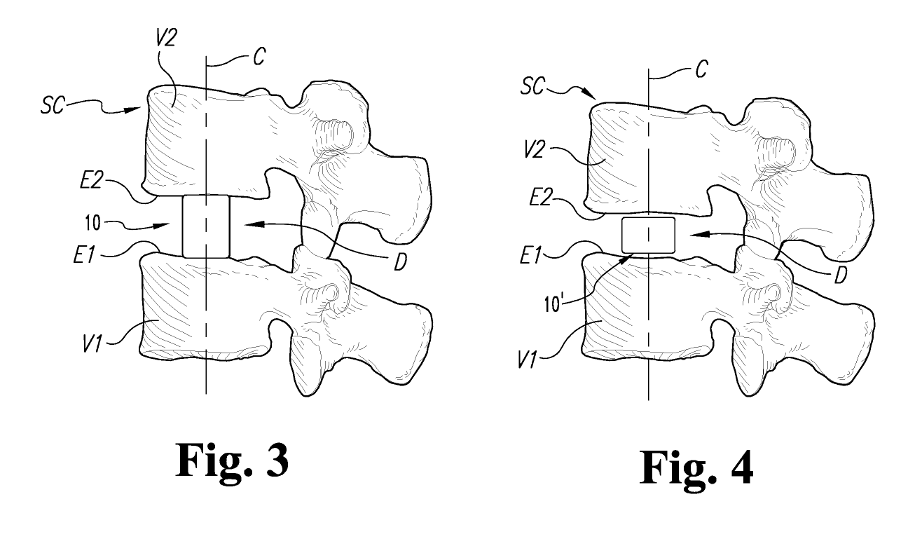

FIG. 1 illustrates a plan view looking caudally toward the axial plane of a vertebral body V1. Spinal interbody implant 10 is positioned on the vertebral endplate E1. Vertebral body V1 is further shown in an anterior view and lateral view in FIGS. 2 and 3, respectively. Vertebral body V1 along with vertebral body V2 and spinal disc space D comprise a level of spinal column segment SC. Implant 10 is positioned in disc space D so its longitudinal axis 12 extends laterally across sagittal plane S and parallel to or generally parallel to coronal plane C. Implant 10 is positioned in disc space D between vertebrae V1 and V2 so that when it is in its implanted orientation it contacts endplates E1 and E2. According to one procedure, implant 10 is positioned into disc space D from a direct lateral approach. As used herein, a “direct lateral approach” is an approach that is parallel or substantially parallel to the coronal plane and thus orthogonal to or substantially orthogonal to the sagittal plane. The term “substantially parallel” means that the approach may vary up to 30 degrees from the parallel to the coronal plane.

FIG. 4 illustrates a lateral view of the spinal column segment SC with implant 10 in a rotated position 10′ to orient its sidewalls toward respective ones of the endplate E1, E2 to provide implant 10′ with a reduced profile in the caudal-cephalad direction and facilitate insertion between endplates E1, E2. In this orientation of implant 10′, it defines an insertion height that is small enough to allow insertion without interference of endplates E1, E2 and without requiring distraction or over-distraction of vertebrae V1, V2, and minimizing or eliminating removal of osteophytes and endplate material. Once implant 10′ is located in the proper medial-lateral position in disc space D, implant 10′ is rotated 90 degrees around its longitudinal axis 12 to orient its superior and inferior bearing surfaces to contact and support respective ones of the endplates E1, E2, such as shown in FIG. 3, and, if necessary, to distract the vertebrae V1, V2 to provide a desired disc space height.

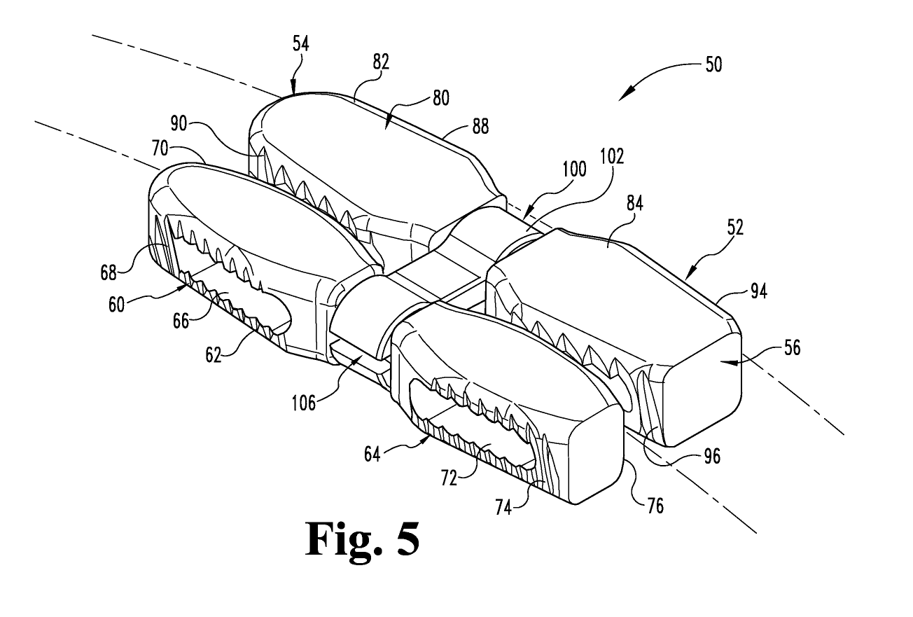

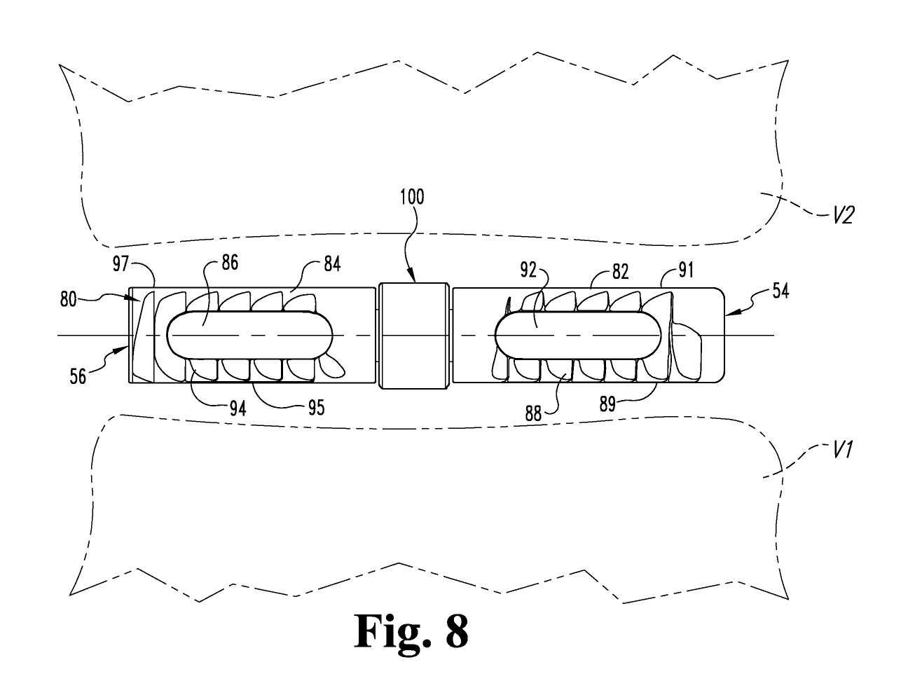

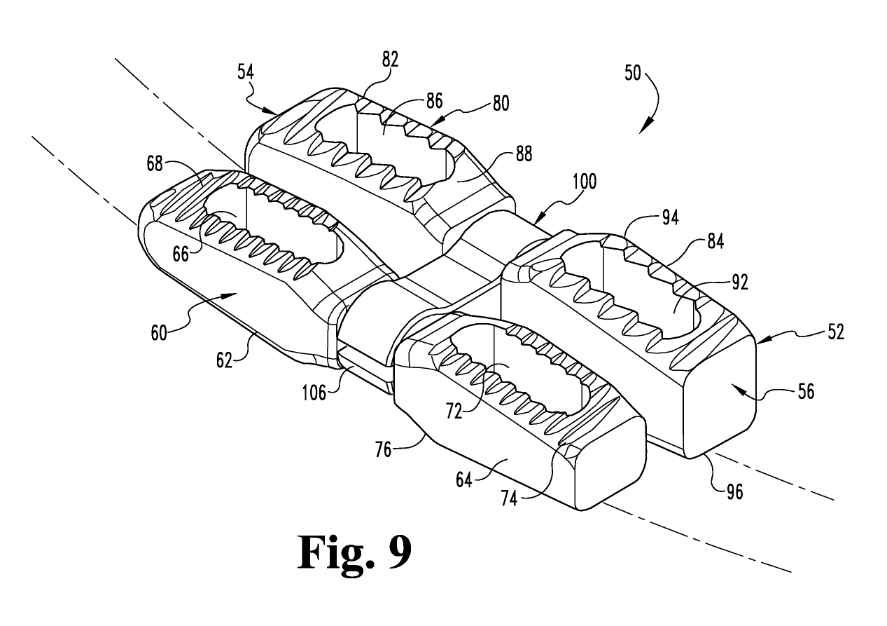

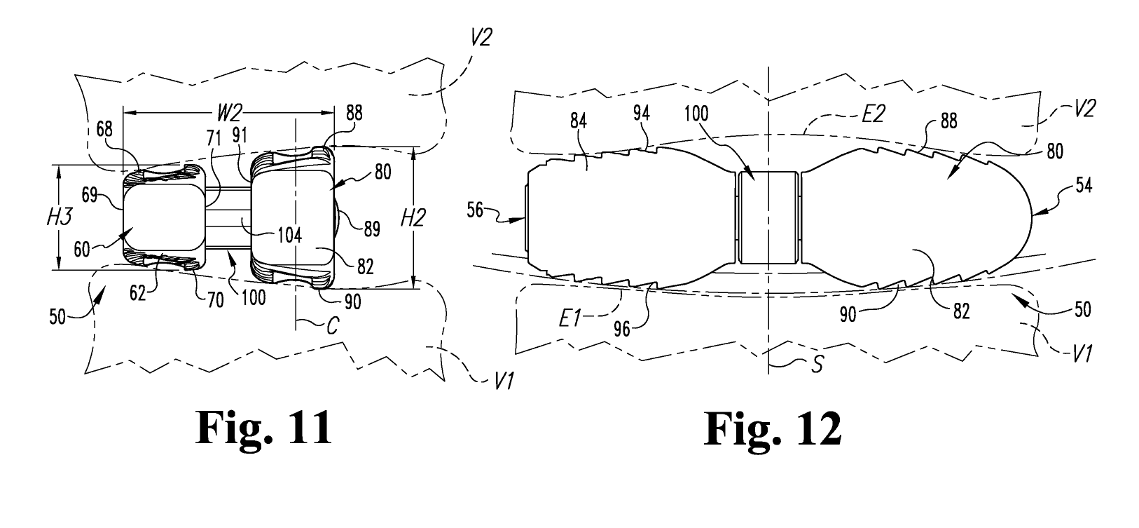

FIGS. 5-8 shows another embodiment of implant 10 designated as implant 50. Implant 50 comprises an elongate body 52 sized to fit within the intervertebral disc space D between adjacent vertebral members V1, V2. In FIGS. 5-8, body 50 is oriented to provide a reduced height H1 to form a low profile orientation for insertion into disc space D so that at least one of the side walls is spaced from the respective adjacent endplate E1, E2. FIGS. 9-12 show implant 10 with body 52 manipulated to an implanted configuration forming a height H2, H3 between opposite vertebral endplate contacting surfaces that is sized to contact endplates E1, E2. Body 52 of implant 50 includes a length L between a leading end 54 and a trailing end 56 sized to extend substantially from a location adjacent the lateral edges of endplates E1, E2 from one side of the disc space D to the opposite side of disc space D. Length L provides bi-lateral support for the vertebrae V1, V2 and contacts the hard cortical bone that extends around the perimeter of endplates E1, E2, such as shown in FIG. 12. Furthermore, body 52 defines a width W1 in the anterior-posterior direction along the sagittal plane when it is in its insertion configuration so that body 52 extends from a location adjacent the anterior edge of endplates E1, E2 to a location spaced posteriorly of coronal plane C, as shown in FIG. 7. When body 52 is manipulated to its implanted configuration as shown in FIG. 11, body 52 defines a smaller width W2 in the anterior-posterior direction along the sagittal plane. The superior and inferior bone engaging surfaces of body 52 contact the endplates E1, E2 at least along the anterior edges of vertebral endplates E1, E2, while the posterior portion of body 52 is located on or adjacent to the coronal plane to provide support along the central axis of the spinal column. As set forth in greater detail below, implant 50 includes one or more inner chambers or cavities through body 52 that may receive bone growth material and/or bone grafts so that bone grows through the body 52 to fuse with the vertebral members V1, V2. Other embodiments contemplate an implant 50 that is solid or without cavities or chambers in the members of the implant, but allows bone growth between and around the members of the implant.

Further details regarding the embodiment of implant 50 will now be discussed. Implant body 52 includes an elongated first member 60 and an elongated second member 80 extending in side-by-side relation through a housing 100. When implanted in disc space D in a direct lateral approach, first member 60 is located anteriorly of second member 80, and housing 60 extends along the sagittal plane S. First member 60 includes a first portion 62 extending along a central longitudinal axis from leading end 54 to housing 100, and a second portion 64 extending along the same central longitudinal axis from housing 100 to trailing end 56. In one embodiment, first and second portions 62, 64 are connected to one another with a neck 63 that extends through housing 100. In another embodiment, first and second portions 62, 64 are separate from one another and separately connected to housing 100. First portion 62 includes a central cavity 66 extending through and opening at superior and inferior bone engaging surface portions 68, 70, respectively. Second portion 64 includes a central cavity 72 extending through and opening at superior and inferior bone engaging surface portions 74, 76, respectively. Each of the superior and inferior bone engaging surface portions 68, 70 of first portion 62 defines an outwardly extending convex curvature extending from leading end 54 to housing 100. In addition, superior and inferior bone engaging surface portions 68, 70 each define an outwardly extending convex curvature between opposite side walls 69, 71, Sidewalls 69, 71 are parallel to one another and extend from superior bone engaging surface portion 68 to inferior bone engaging surface portion 70 and from housing 100 to leading end 54. Each of the superior and inferior bone engaging surface portions 74, 76 of second portion 64 defines an outwardly extending convex curvature extending from trailing end 56 to housing 100. In addition, superior and inferior bone engaging surface portions 74, 76 define an outwardly extending convex curvature between opposite side walls 75, 77. Sidewalls 75, 77 are parallel to one another and extend from superior bone engaging surface portion 74 to inferior bone engaging surface portion 76 and from housing 100 to trailing end 56. In the insertion orientation of implant 50, first member 60 is rotated about its central longitudinal axis in housing 100 so that parallel sidewalls 69, 71 and parallel sidewalls 75, 77 are oriented toward respective ones of the endplates E1, E1 to form the reduced height H1 for insertion of implant 50 into disc space D.

Second member 80 includes a first portion 82 extending along a central longitudinal axis thereof from leading end 54 to housing 100, and a second portion 84 extending along the same central longitudinal axis from housing 100 to trailing end 56. In one embodiment, first and second portions 82, 84 are connected to one another with a neck 83 that extends through housing 100. In another embodiment, first and second portions 82, 84 are separate from one another and separately connected to housing 100. First portion 82 includes a central cavity 86 extending through and opening at superior and inferior bone engaging surface portions 88, 90, respectively. Second portion 84 includes a central cavity 92 extending through and opening at superior and inferior bone engaging surface portions 94, 96, respectively. Each of the superior and inferior bone engaging surface portions 88, 90 of first portion 82 defines an outwardly extending convex curvature extending from leading end 54 to housing 100. In addition, superior and inferior bone engaging surface portions 88, 90 of first portion 82 each define an outwardly extending convex curvature between opposite side walls 89, 91. Sidewalls 89, 91 are parallel to one another and extend from superior bone engaging surface portion 88 to inferior bone engaging surface portion 90 and from housing 100 to leading end 54. Each of the superior and inferior bone engaging surface portions 94, 96 of second portion 84 defines an outwardly extending convex curvature extending from trailing end 56 to housing 100. In addition, superior and inferior bone engaging surface portions 94, 96 define an outwardly extending convex curvature between opposite side walls 95, 97. Sidewalls 95, 97 are parallel to one another and extend from superior bone engaging surface portion 94 to inferior bone engaging surface portion 96 and from housing 100 to trailing end 56. In the insertion orientation of implant 50, second member 80 is rotated in housing 100 about its central longitudinal axis so that parallel sidewalls 89, 91 and parallel sidewalls 95, 97 are oriented toward respective ones of the endplates E1, E1 to form the reduced height H1 for insertion of implant 50 into disc space D.

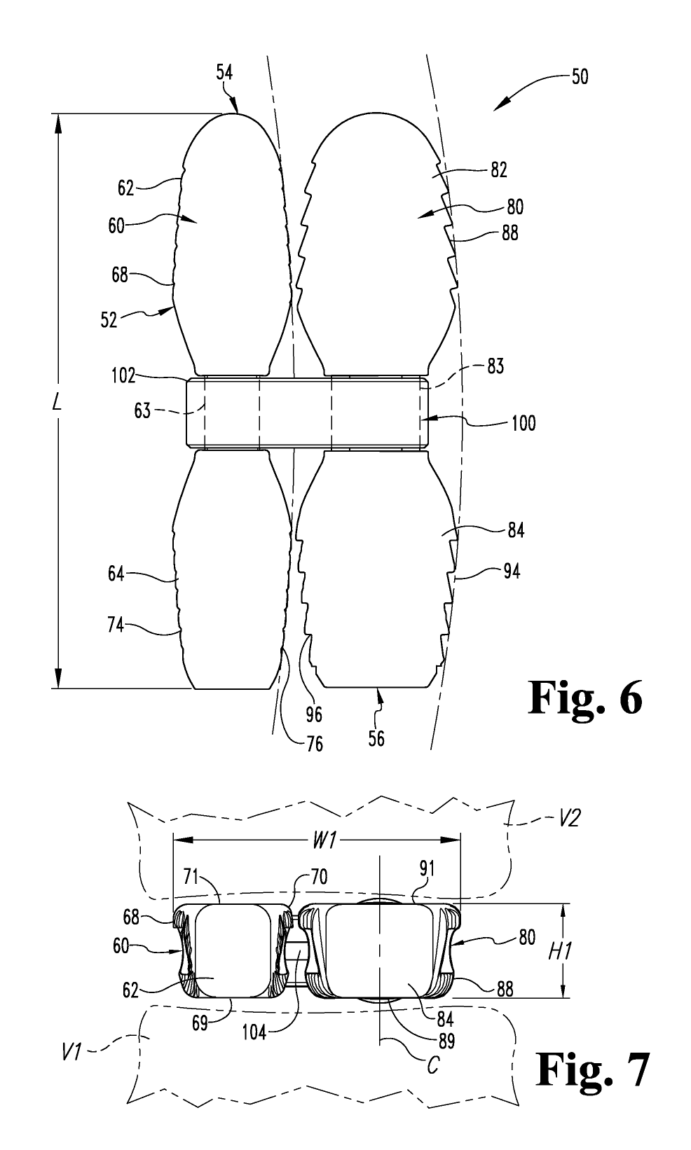

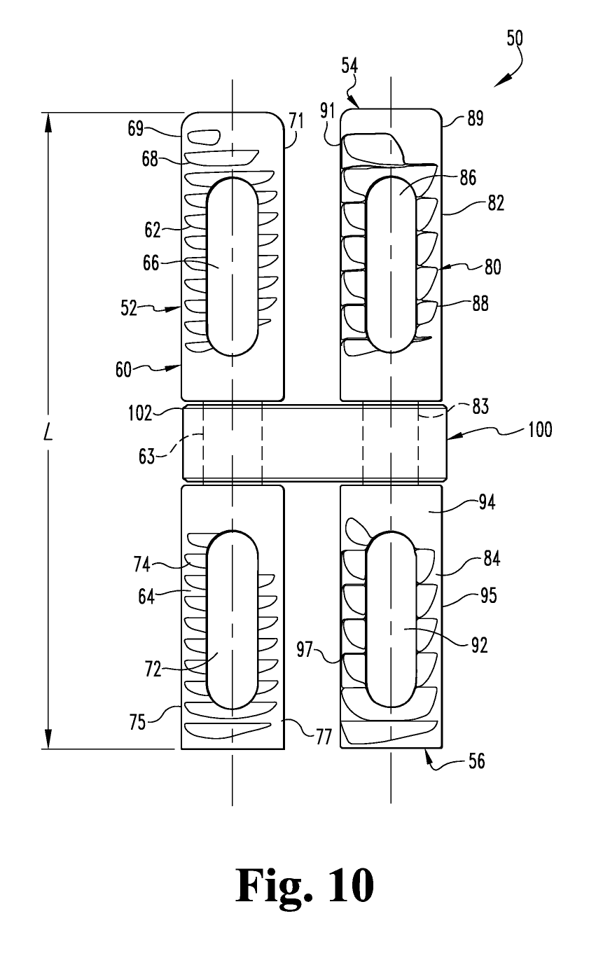

First and second members 60, 80 are coupled to housing 100 and extend from housing 100 in side-by-side and spaced relation. Housing 100 maintains their spaced relation from one another along length L in both the initial insertion configuration and in the implanted configuration. As shown in FIGS. 6 and 7, in the initial insertion configuration, inferior bone engaging surface portions 70, 76 of first member 60 are oriented toward and face inferior surface portions 90, 96 of second member 80. When implant 50 is located in the desired position in disc space D, first and second members 60, 80 are each rotated about its respective central longitudinal axis in either a clockwise or counter-clockwise direction so that inner side walls 71, 77 of first member 60 are located adjacent to and face inner side walls 91, 97 of second member 80 and position the inferior and superior bone engaging surface portions in contact with the respective vertebral endplates E1, E2, as shown in FIGS. 10-11.

Housing 100 includes a body 102 that is elongated in a direction extending between the anterior and posterior sides of implant body 52 to define a length that is substantially the same as width W2 of implant 50. Housing 100 also defines a height that is less than height H2, H3 of implant 50 so that housing 100 does not contact the endplates E1, E2 in the implanted orientation of implant 50. Housing 100 defines a cavity 104 for rotatably receiving necks 63, 83 therein. One side of body 102 defines a slot or opening 106 that allows body 102 to be flexed open to receive first and second members 60, 80 therein when implant 50 is assembled, and then allow body 102 to be closed to secure first and second members 60, 80 in housing 100. Housing 102 frictionally engages necks 63, 83 to maintain first and second members 60, 80 in spaced relation from one another while allowing first and second members 60, 80 to be rotated from the initial insertion orientation to the implanted orientation in the disc space D. The arrangement of implant 50 with first and second members 60, 80 in side-by-side relation allows the overall height of implant 50 to be minimized in its initial insertion than would be possible with a single member, while providing a greater width in the implanted configuration to increase stability of implant 50 and the surface area contact with the adjacent endplates E1, E2. In addition, in another embodiment, housing 100 allows first and second members 60, 80 to be moved toward and away from one another to adjusting the spacing between the adjacent inner side walls so that the positioning of first and second members 60, 80 can be adjusted in situ in the disc space to optimize the fit with the adjacent endplates E1, E2.

In the implanted configuration, the aligned superior bone engaging surface portions 68, 88 of first portions 62, 82 and aligned superior bone engaging surface portions 74, 94 of second portions 64, 84 form a continuously curved convex outer profile in or along the sagittal plane, and the aligned inferior bone engaging surface portions 70, 90 of first portions 62, 82 and aligned inferior bone engaging surface portions 76, 96 of second portions 64, 84 also form a continuously curved convex outer profile. The maximum height H2 along the posteriorly facing side walls 91, 97 is greater than the maximum height H2 along the anteriorly facing side walls 69, 75 to establish lordosis correction between endplates E1, E2 of vertebrae V1, V2, and also to allow second member 80 to contact the endplates E1, E2 along coronal plane C where the concavity of endplates E1, E2 is greatest. The superior and inferior bone engaging surface portions of first and second members 60, 80 may also include grooves or recesses that interrupt the convex outer profile to form vertebral endplate engaging structures across the width of first and second members 60, 80, and also to accommodate the central cavities extending through the implant portions 62, 64, 82, 84. The bone engagement structures can comprise grooves, recesses, ridges, serrations, knurlings, spikes, roughened surfaces, or smooth surfaces for engaging the endplates E1, E2 of the adjacent vertebral members V1, V2. As illustrated, the bone engagement structures extend in a direction between the adjacent sidewalls of the respective member 60, 80. Other embodiments contemplate engagement structures that extend in a direction between leading end 54 and trailing end 56, or obliquely to the length and/or width of first and second members 60, 80.

The leading end 54 of implant 50 is rounded or tapered between the respective superior and inferior bone engaging surface portions of first and second members 60, 80 so that the body 52 conforms to the anatomy of the endplates E1, E2 in the disc space. A rounded leading 54 can also distract the adjacent vertebral members V1, V2 as the body 52 is inserted in a collapsed disc space D if implant 50 is oriented and inserted in its implanted configuration. Trailing end 56 can be flat and solid as shown. Other embodiments contemplate that the trailing end 56 can include one or more holes, threaded openings, slots or other structure of one or both of first and second members 60, 80 to facilitate engagement with an insertion instrument. In addition, first member 60 can define a length between its leading and trailing end portions that is the same as the length of second member 80 between its leading and trailing end portions. In another embodiment, the length of first member 60 is less than the length of second member 80 so that when first member 60 is positioned in the anterior portion of the disc space D, its leading and trailing end portions do not overhang the lateral edges of the vertebral endplates E1, E2, while second member 80 is positioned medially-laterally in the central portion of the disc space D and thus can have a greater length than first member 60 without projecting from or overhanging the endplates E1, E2.

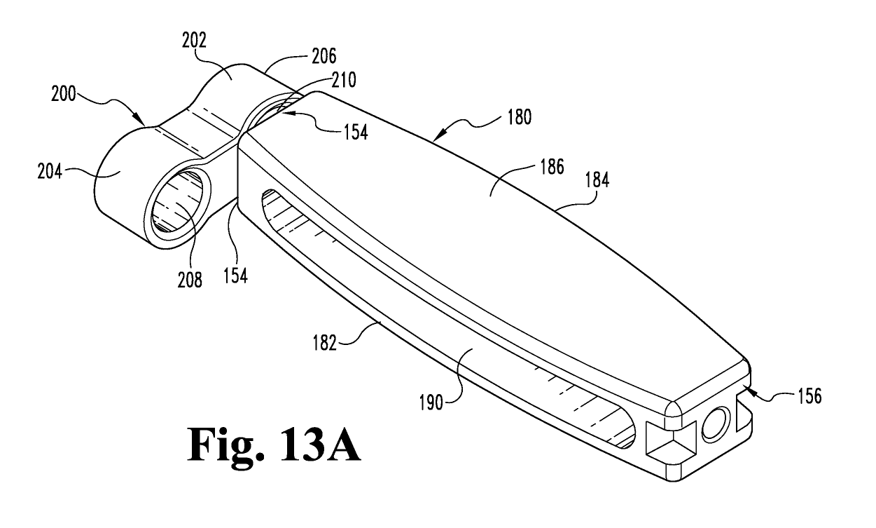

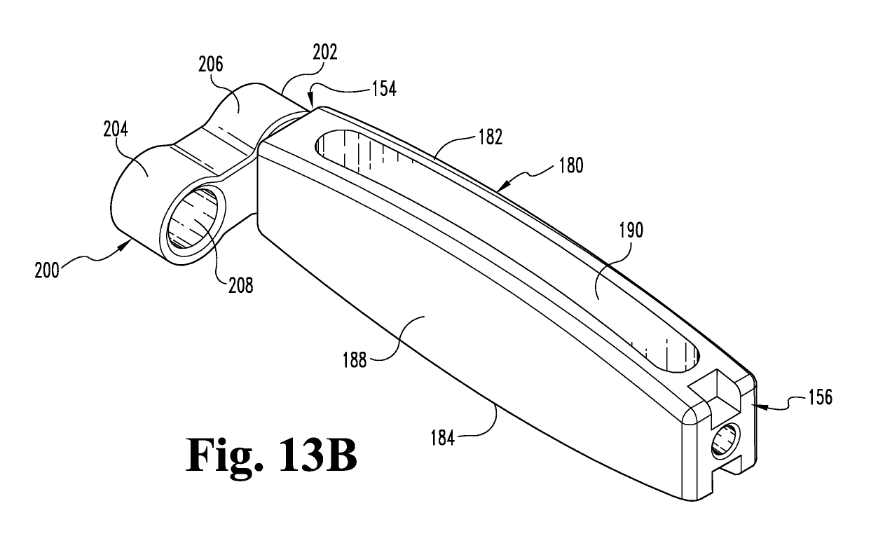

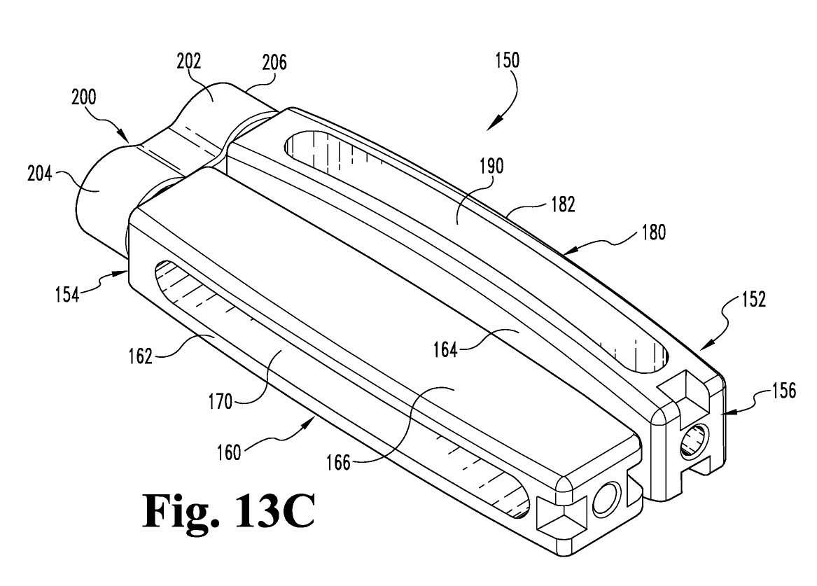

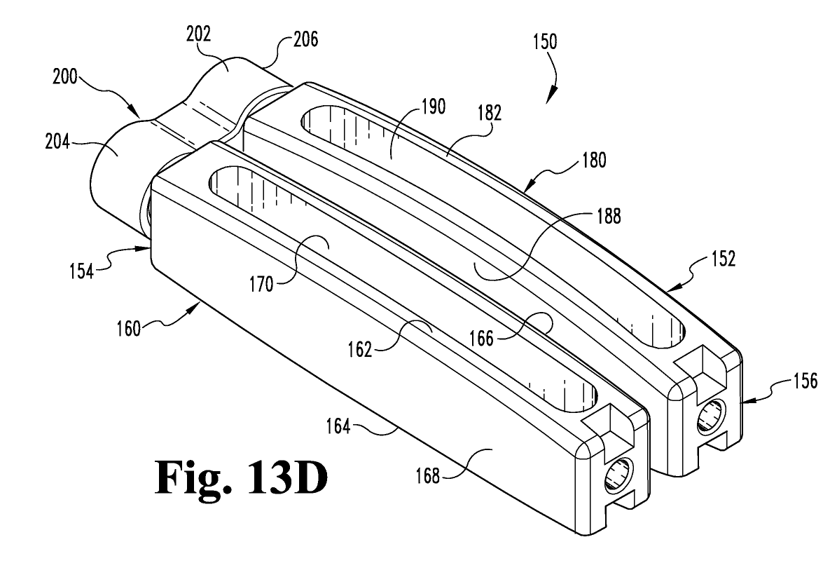

Referring to FIGS. 13A-13D, another embodiment of implant is shown and designated as implant 150. Implant 150 is similar to implant 50 in that it includes a body 152 with a first member 160, a second member 180, and a housing 200 that connects first and second members 160, 180 in side-by-side relation. However, housing 200 is connected to the leading end 154 of first and second members 160, 180 so that each of first and second members 160, 180 extend substantially only in one direction from housing 200. Housing 200 includes a body 202 having a pair of connected nodes 204, 206 that define receptacles 208, 210, respectively. The leading end 154 of first and second implant members 160, 180 is positioned in respective ones of receptacles 208, 210 to maintain first and second members 160, 180 in spaced relation to one another.

First and second members 160, 180 are substantially identical to one another in the illustrated embodiment, although first and second members 160, 180 that substantially differ from one another are not precluded. First member 160 includes a superior bone engaging surface 162 and an opposite inferior bone engaging surface 164 that extend between leading and trailing ends 154, 156. First member 160 also includes opposite parallel sidewalls 166, 168 that extend between superior and inferior bone engaging surfaces 162, 164 and between leading and trailing ends 154, 156. First member 160 includes a cavity or chamber 170 extending between and opening at superior and inferior bone engaging surfaces 162, 164. Superior and inferior bone engaging surfaces 162, 164 are convexly curved between leading end 154 and trailing end 156 to conform to the concave curvature of the endplates E1, E2 when first member 160 is positioned in disc space D. In the illustrated embodiment, superior and inferior bone engaging surfaces 162, 164 are smooth, although providing bone engagement features along these surfaces is also contemplated. Second member 180 includes a superior bone engaging surface 182 and an opposite inferior bone engaging surface 184 that extend between leading and trailing ends 154, 156. First member 180 also includes opposite parallel sidewalls 186, 188 that extend between superior and inferior bone engaging surfaces 182, 184 and between leading and trailing ends 154, 156. First member 180 includes a cavity of chamber 190 extending between and opening at superior and inferior bone engaging surfaces 182, 184. Superior and inferior bone engaging surfaces 182, 184 are convexly curved between leading end 154 and trailing end 156 to conform to the concave curvature of the endplates E1, E2 when second member 180 is positioned in disc space D. In the illustrated embodiment, superior and inferior bone engaging surfaces 182, 184 are smooth, although providing bone engagement features along one or more of these surfaces is also contemplated.

In one procedure involving insertion of implant 150 into disc space D, second member 180 is connected at its leading end 154 to housing 200. Second member 180 is rotated to a reduced profile orientation with side walls 186, 188 oriented toward endplates E1, E2 and then inserted along with housing 200 into the disc space, as shown in FIG. 13A. Second member 180 is then rotated to an implanted orientation so that superior and inferior bone engaging surfaces 182, 184 contact the respective endplates E1, E2. First member 160 is then inserted in a reduced profile orientation where side walls 166, 168 are oriented toward respective ones of the endplates E1, E2, and guided into the disc space D to engage its leading end to housing 200 as shown in FIG. 13C. First member 160 is then rotated while engaged to housing 200 so that superior and inferior bone engaging surfaces 162, 164 contact the respective vertebral endplate E1, E2. The sequential insertion and rotation of first and second members 160, 180 allows placement of first and second members 160, 180 in closer relation to one another since first and second members are not simultaneously connected to housing 200. In their reduced profile orientation, the height between the superior and inferior bone engaging surfaces of first and second members 160, 180 requires a greater spacing between first and second members 160, 180 than when one of the first and second members 160, 180 is rotated to its implanted orientation. In addition, it is also contemplated that first member 160 can be first inserted into the disc space D and then rotated to its implanted orientation, and then second member 180 inserted and connected to housing 200 for rotation to its implanted orientated.

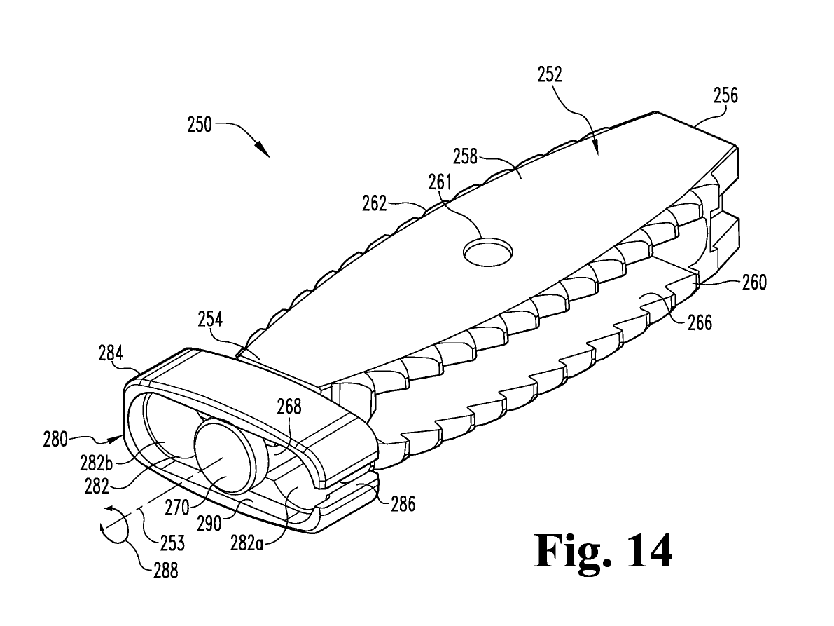

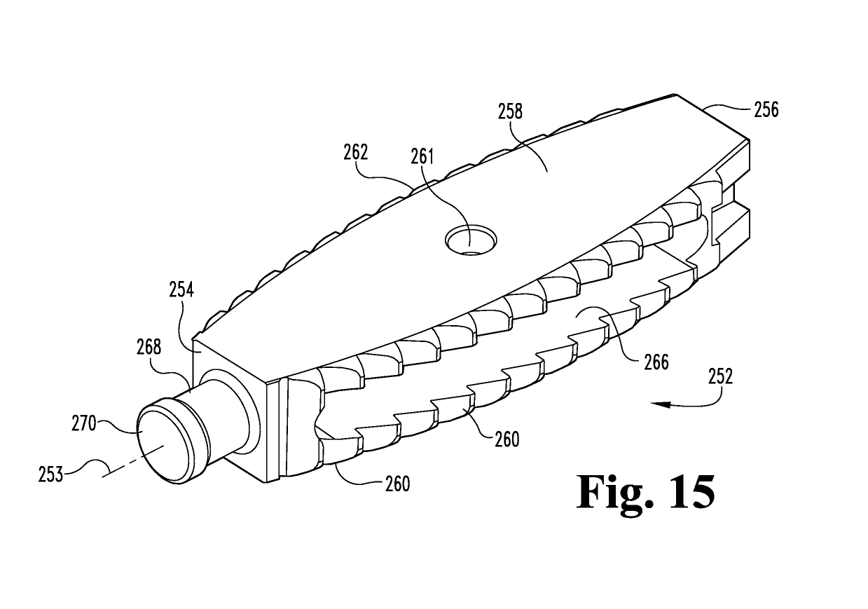

Referring now to FIG. 14, there is shown an implant 250 that includes a single elongated body 252 attached to a housing 280. Elongated body 252, shown in isolation in FIG. 15, extends on a central longitudinal axis 253 between a leading end 254 and an opposite trailing end 256. Body 252 includes opposite parallel sidewalls 258, 260 that extend from leading end 254 to trailing end 256. Implant body 252 also includes a superior bone engaging surface 262 and an opposite inferior bone engaging surface 264. Bone engaging surfaces 260, 262 each extend between side walls 258, 260 and also between leading and trailing ends 254, 256. A central cavity 266 extends through and opens at bone engaging surfaces 260, 262. Bone engaging surfaces 260, 262 may be smooth or include ridges, spikes, teeth or other bone engaging structure extending therealong. In addition, side walls 258, 260 each includes a hole 261 (only one shown) extending therethrough into central cavity 266.

Implant body 252 also includes a neck 268 extending outwardly from leading end 254 that is received in a receptacle 282 of housing 280. Neck 268 includes a head 270 at its outer end that retains implant body 252 in housing 280. Housing 280 includes a C-shaped body 284 with receptacle 282 extending through opposite sides thereof, and a slot 286 at one end thereof that allows the receptacle to be widened to receive neck 268 and head 270. In FIGS. 14 and 15, body 252 is shown in an insertion orientation where side walls 258, 260 are oriented to face endplates of the adjacent vertebrae. Once implant 250 is positioned in the disc space in this insertion orientation, body 252 is rotated about central longitudinal axis 253 as indicated by arrow 288 so that bone engaging surfaces 260, 262 contact the endplates of the adjacent vertebrae. Body 252 can be located in the center of receptacle 282 as shown in FIG. 14, or moved laterally (anteriorly or posteriorly if body 252 is positioned along the coronal plane) in receptacle 282 to one of the anterior or posterior receptacle portions 282a, 282b. The center, anterior and posterior receptacle portions of receptacle 282 can be compartmentalized with ribs or projections that extend partially into receptacle 282 to form discrete receptacle portions, but that allow the receptacle portions to be in communication with one another so that neck 268 can be moved from one portion to the other without removing neck 268 and head 270 from housing 280. In addition, housing 280 defines an internal lipped region 290 around receptacle 282 that receives head 270. Movement of implant body 252 along longitudinal axis 253 is prevented by contact of leading end 254 with one side of housing 280 and contact of head 270 with housing 280 in lipped region 290. In still other embodiment, one or more additional implant bodies are engaged to housing 280 in side-by-side relation to implant body 252.

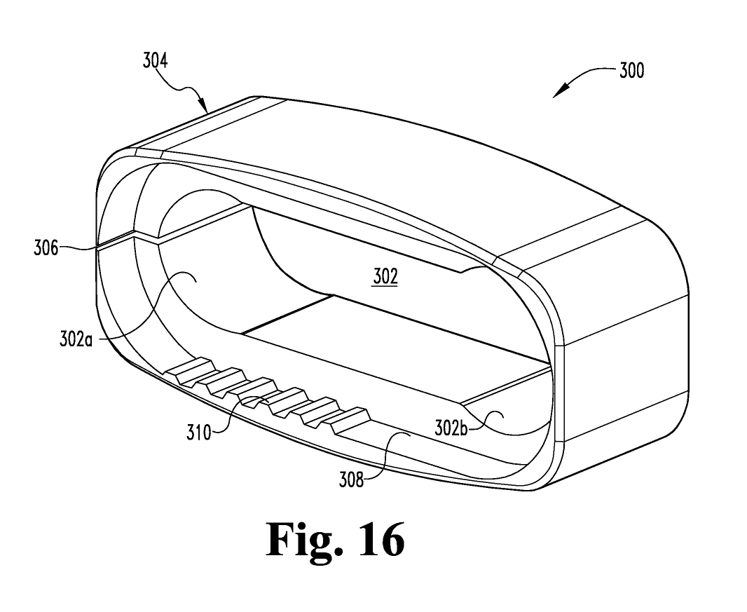

Referring now to FIG. 16, there is shown another embodiment housing 300. Housing 300 is similar to the other embodiment housings discussed herein, but includes a gearing mechanism to assist in moving an implant member across its receptacle. Housing 300 includes an oval or C-shaped body 304 with a receptacle 302 that extends through and opens at opposite sides of body 304. Body 304 also includes a slot 306 at one end thereof that allows body 304 to be flexed to widen receptacle 302 to accommodate placement of a portion of the implant body therein, and to prevent the implant body from binding in housing 300 as the implant body is rotated. Body 304 include a portion of its length that narrows in width between the sides in which receptacle 302 opens and toward one of its ends to accommodate placement in a disc space, as discussed further below. Receptacle 302 includes a first portion 302a located at one end thereof and a second portion 302b located at the opposite end of receptacle 302. Second portion 302b is enlarged relative to the remaining portion of receptacle 302 to facilitate insertion of an implant body therein when the implant body is in its initial insertion orientation. In addition, housing body 304 includes an internal lipped region 308 that extends around receptacle 302. Internal lipped region 308 also includes gear teeth 310 extending therealong that are recessed away from receptacle 302 and extend from receptacle portions 302b to receptacle portion 302a. As discussed further below, gear teeth 310 engage corresponding teeth on a head of the implant member to assist in moving the implant member from receptacle portion 302b to receptacle portion 302a as the implant body is rotated about its central longitudinal axis.

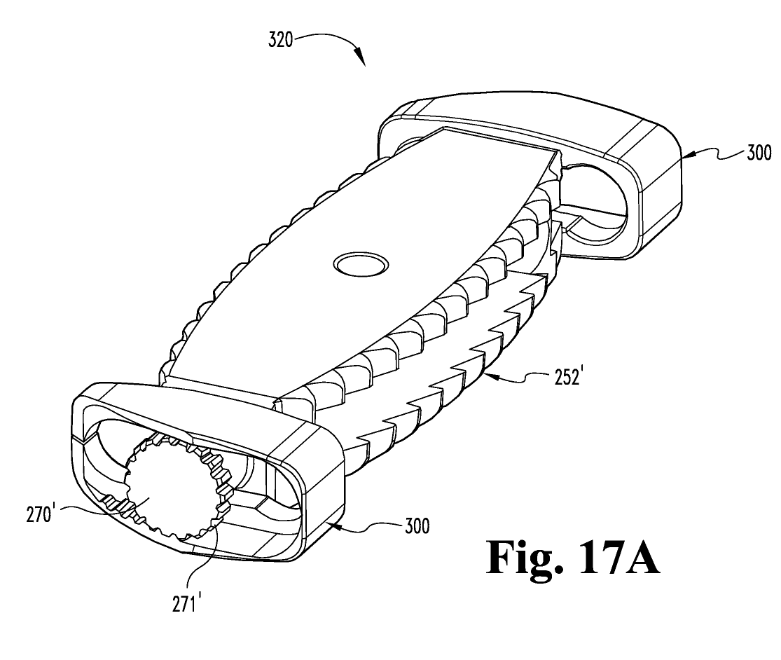

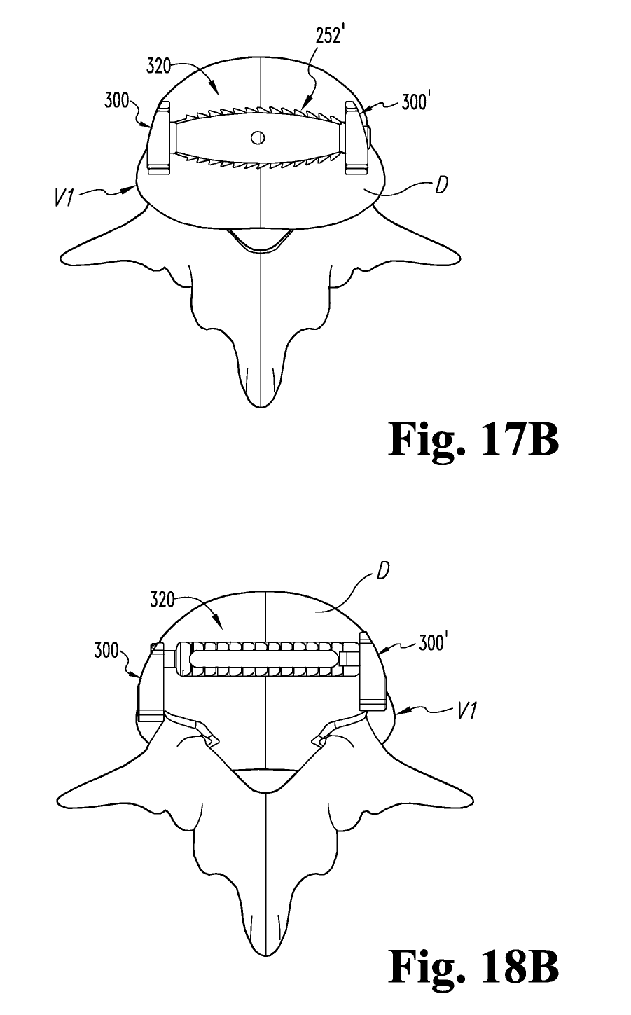

FIGS. 17A-17B show a spinal interbody implant 320 that includes an elongate implant body 252′ connected with first and second housings 300, 300′ at the opposite ends of implant body 252′. Implant body 252′ can be, for example, identical to implant body 252 discussed above or any of the other implant bodies discussed herein. However, implant body 252′ includes a modified head 270′ at one end of body 252′ and an identical second neck 268 and modified head 270′ at the opposite end of body 252′ (not shown) to engage the second housing 300′. Housings 300, 300′ are mirror images of one another, and housing 300′ include gear teeth like gear teeth 310 of housing 300. Modified heads 270′ include teeth 271′ extending around the circular perimeter thereof that engage and mesh with gear teeth 310 of the respective housing 300, 300′.

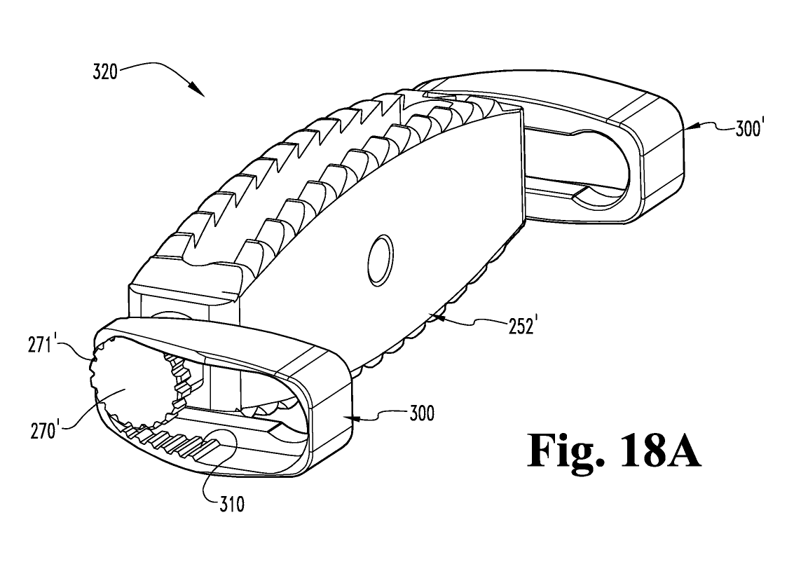

In the insertion orientation of implant 320 shown in FIGS. 17A-17B, implant body 252′ is positioned in or adjacent to receptacle portion 302b of housings 300, 300′ and inserted into disc space D with the side walls 258, 260 positioned to face the respective vertebral endplates when in disc space D. Housings 300, 300′ are located adjacent the lateral edges of vertebra V1 and the other adjacent vertebra when implant 310 is positioned in a lateral approach along the coronal plane, as shown in FIG. 17B. The tapered anterior portion of body 304 allows positioning of housings 300, 300′ as far anteriorly as possible while preventing housings 300, 300′ from projecting laterally outwardly from disc space D. Implant body 252′ is then rotated about its central longitudinal axis to an implanted orientation shown in FIGS. 18A-18B. As implant body 252′ is rotated, the teeth of head 270′ engage with the gear teeth 310 of the corresponding housing 300, 300′ to facilitate rotation of implant body 252′ to its implanted orientation, and to advance implant 252′ in the disc space as far anteriorly as possible relative to the vertebrae and the implanted positions of housings 300, 300′. Implant member 252′ can remain the sole implant member between housings 300, 300′. Alternatively, in procedures where side-by-side implant members are desired, a second implant member can be located in receptacle portions 302b of housing 300, 300′.

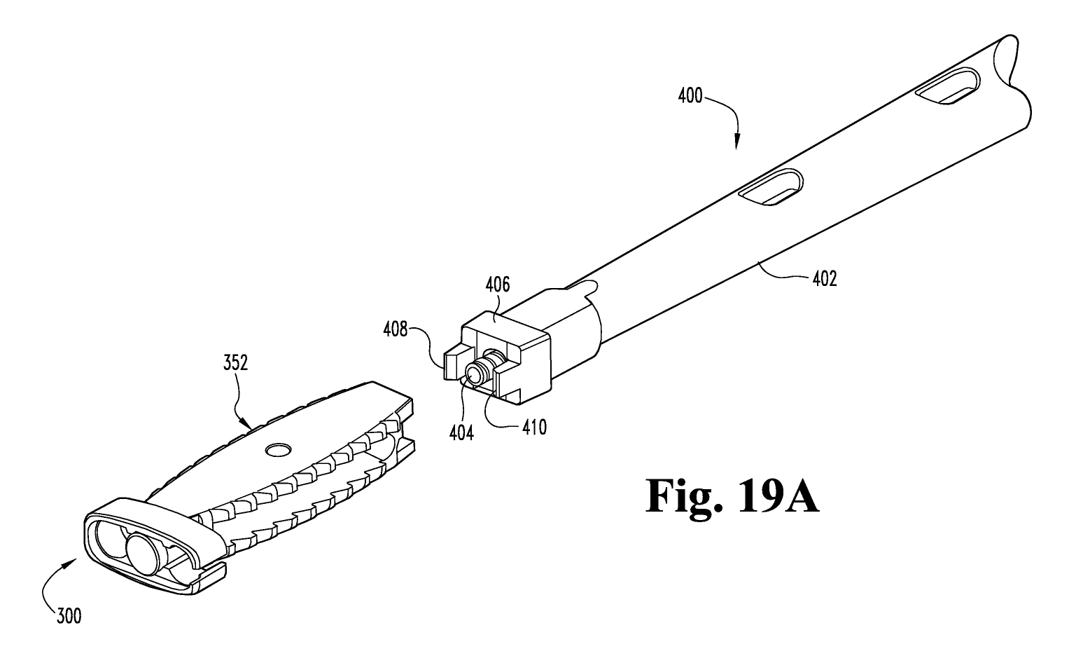

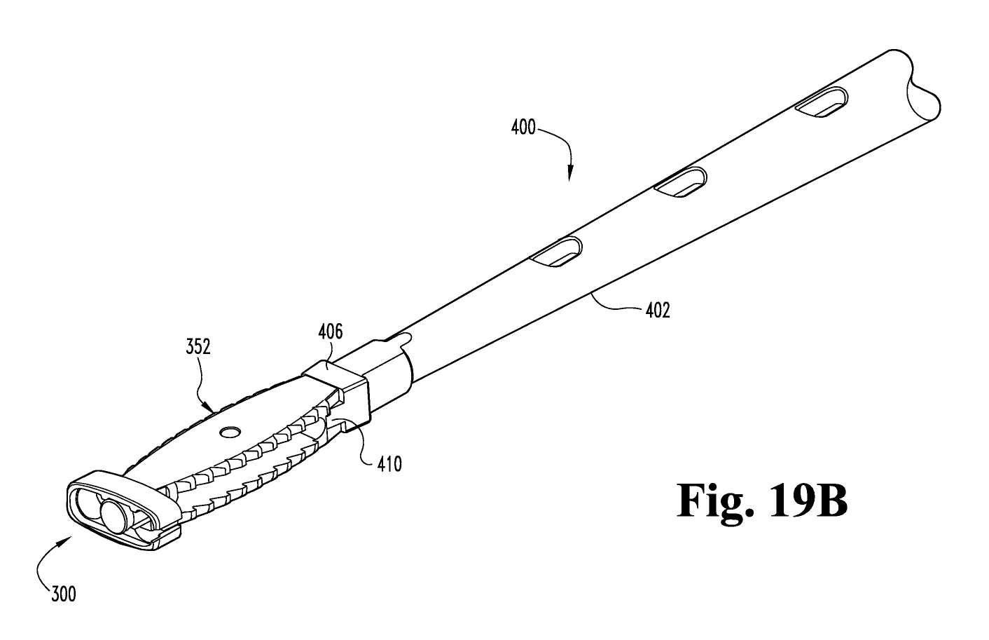

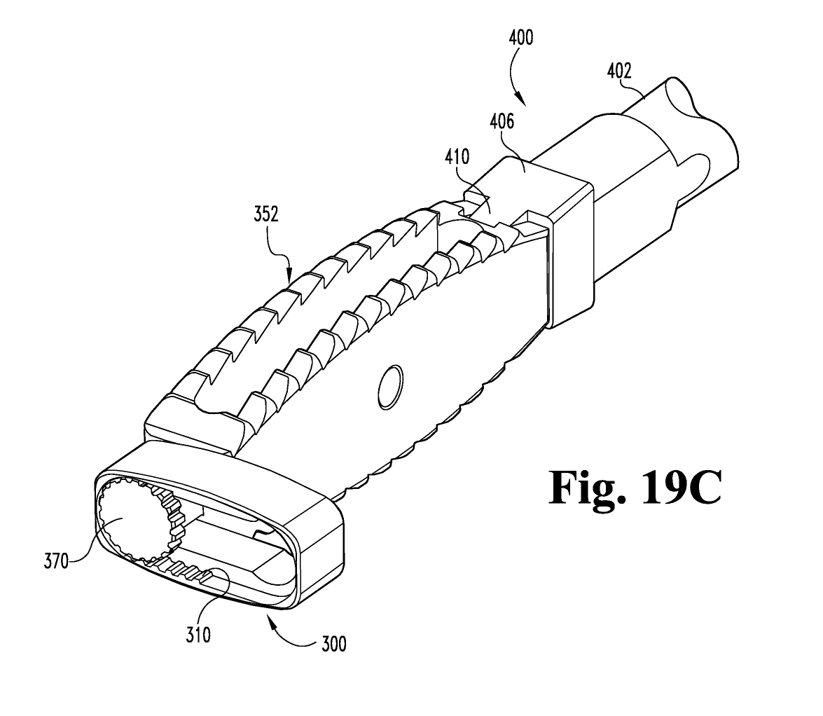

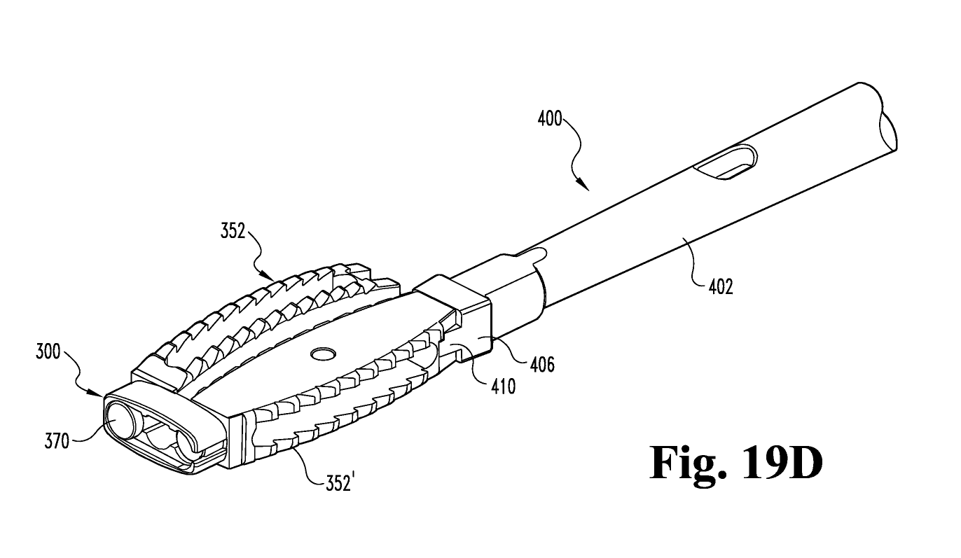

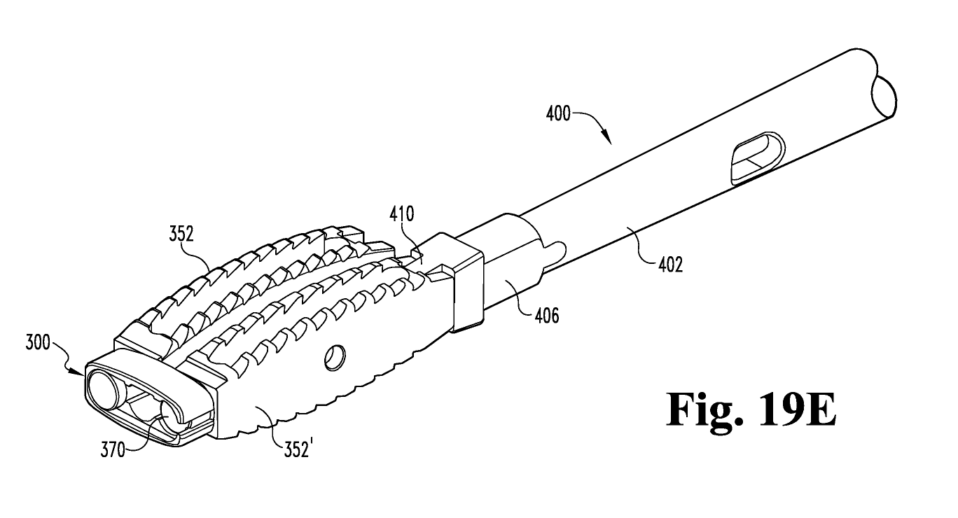

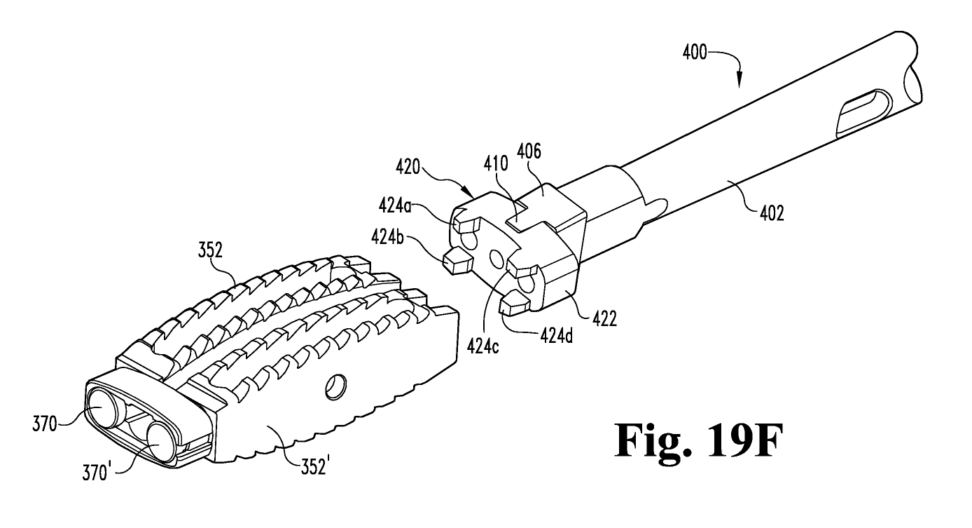

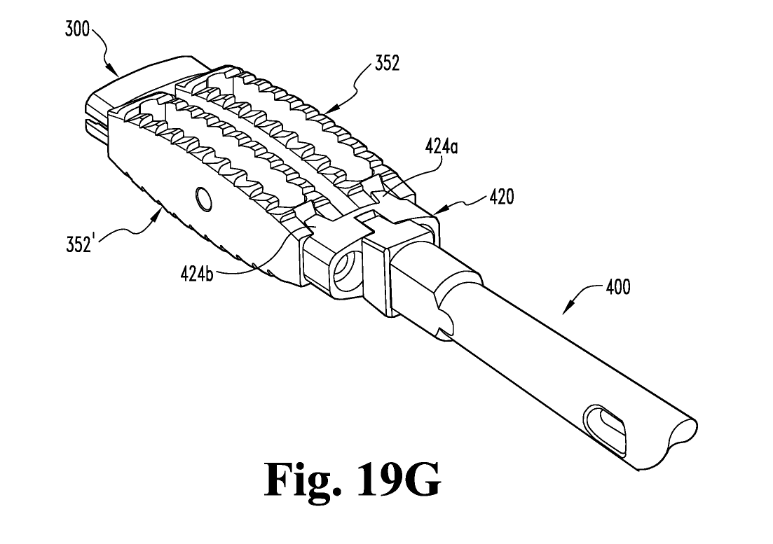

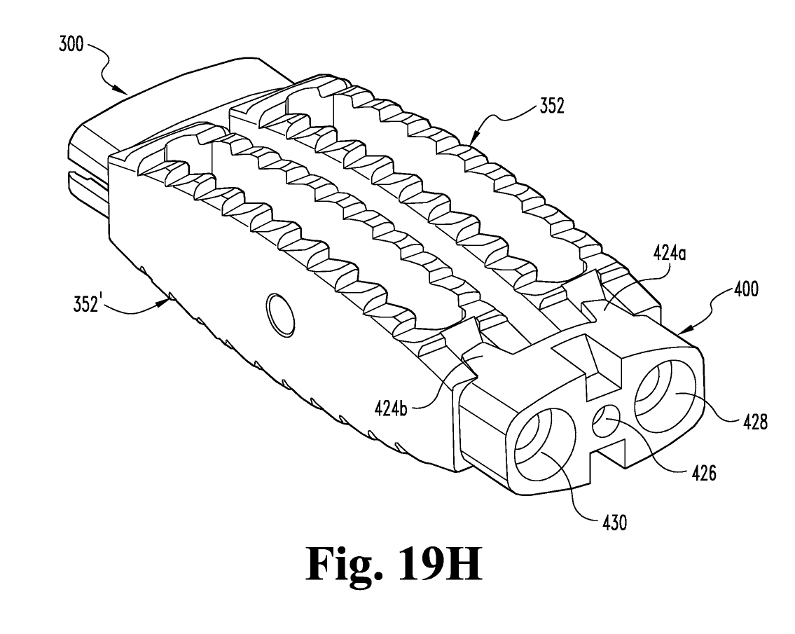

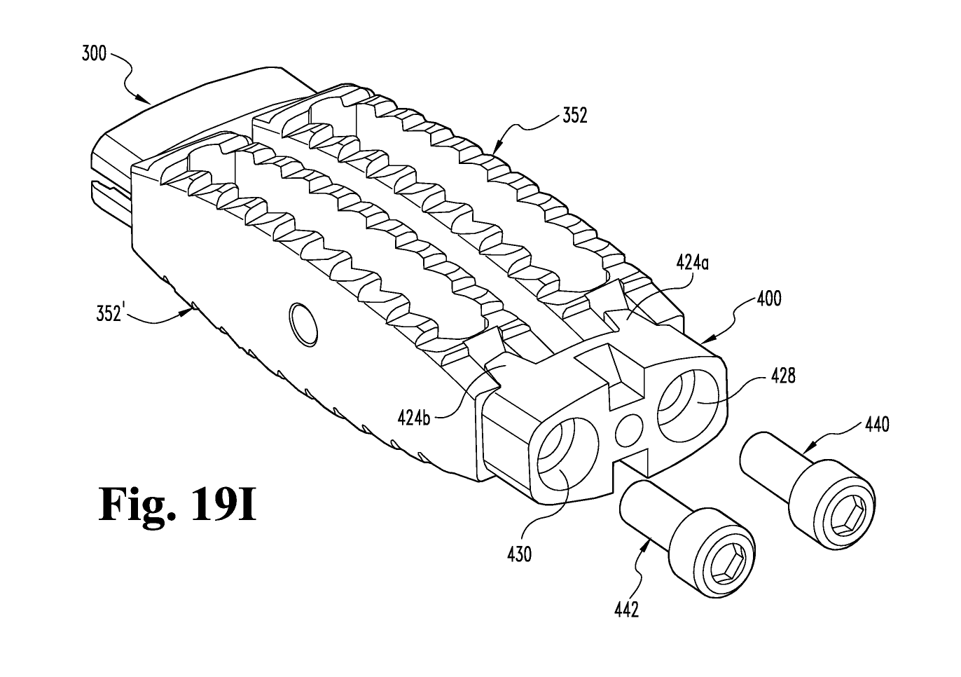

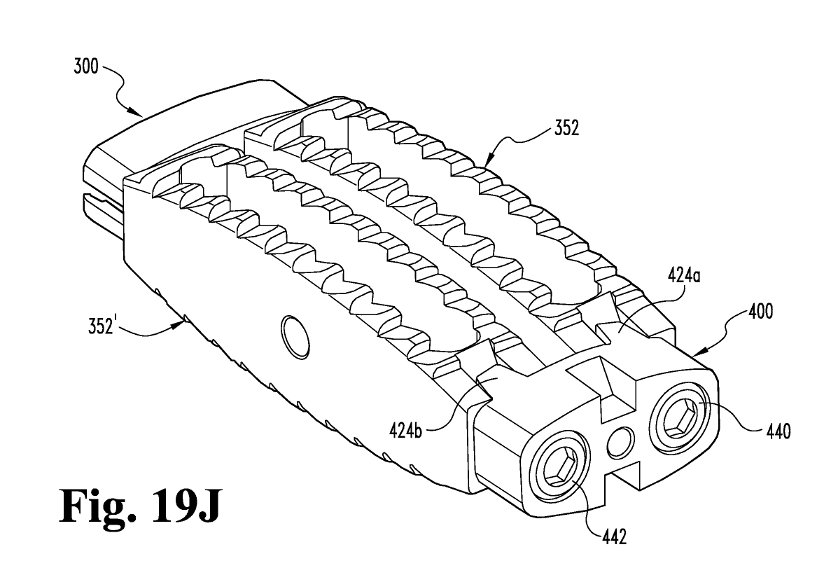

FIGS. 19A-19J show an insertion technique along with portions of instruments associated with inserting a pair of elongate implant implants in side-by-side relation in the disc space. In FIG. 19A there is shown housing 300 connected with an elongate implant member 352 having a leading end engaged to housing 300. Housing 300 is shown without gear teeth 310 in FIGS. 19A-19B and 19D-19F for purposes of clarity, but it is understood that housing 300 includes gear teeth 310 in one embodiment. Other embodiments contemplate that any of the housing embodiments discussed herein could be employed in the insertion technique. Implant member 352 can be the same or similar to implant member 252 discussed above, but includes a modified head 370 at only one end thereof, with head 370 including teeth extending around the perimeter thereof to mesh with the gear teeth 310 of housing 300. Implant member 352 is engaged to housing 300 in its initial insertion orientation prior to implantation. Also shown is an inserter 400 that includes an elongated sleeve 402 and a central shaft 404 housed within sleeve 402. Shaft 404 is engageable to an opening or receptacle in the trailing end of implant member 352 via a threaded connection, interference fit, or other suitable connection. Sleeve 404 includes a head 406 at its distal end with upper and lower distally extending flanges 408, 410 that are received in grooves of implant member 352. The grooves are recessed into the upper and lower bone engaging surfaces of implant member 352 and open at the trailing end of implant member 352 to receive respective ones of the flanges 408, 410, as shown in FIG. 19B. Using inserter 400, housing 300 and implant member 352 are inserted together into the disc space with implant member 352 in its insertion orientation, and then inserter 400 is rotated as indicated by arrow 412 in FIG. 19C to rotate implant member 352 around its central longitudinal axis in housing 300 to its implantation orientation. In procedures utilizing a direct lateral approach, rotation of implant member 352 translates implant member 352 anteriorly in housing 300 and moves implant member 352 toward the anterior side of the vertebrae to first restore the anterior height of the disc space.

Referring now to FIG. 19D, a second implant member 352′ is inserted that is similar to implant member 352, but can include a greater height in its implanted orientation than implant member 352 to accommodate its placement more toward the center of the disc space, such as discussed above with respect to interbody implant 50 and its implant members 60, 80. Inserter 400 is engaged to the trailing end of second implant member 352′ in a manner like that discussed above with respect to implant member 352. Second implant member 352′ is positioned in the disc space in its insertion orientation to engage its leading end with housing 300, and then rotated to its implanted orientation in housing 300 as shown in FIG. 19E. The head 370′ of second implant member 352′ need not be provided with teeth since second implant member 352′ will not be moved along the length of the receptacle of housing 300, although providing such teeth on the head of implant member 352′ is not precluded.

In FIG. 19F, inserter 400 is shown engaged to a cap 420 that is to be engaged to the trailing ends of implant members 352, 352′. Cap 420 engages and maintains the relative spacing between implant members 352, 352′ to provide a stable construct. Cap 420 includes a body 422 and four flanges 424a, 424b, 424c, 424d that are received in respective ones of the grooves in the superior and inferior bone engaging surfaces of implants members 352, 352′, as shown in FIG. 19G. Body 422 also includes a central hole 426, and a pair of lateral holes 428, 430 on opposite sides of central hole 426, as shown in FIG. 19H. Central hole 426 receives the shaft 404 of inserter 400 with flanges 408, 410 positioned on the upper and lower sides of body 422. Lateral holes 428, 420 align with holes or bores in the trailing ends of respective ones of implant members 352, 352′ so that fasteners 440, 442 (FIG. 19I) can be placed through respective ones of the lateral holes 428, 430 to secure cap 420 to implant members 352, 352′, as shown in FIG. 19J. Lateral holes 428, 430 each include a counter bore so that fasteners 440, 442 are recessed or flush with the outer surface of cap 420 in the final construct of the interbody spinal implant.

Materials for the implants disclosed herein can be chosen from any suitable biocompatible material, such as titanium, titanium alloys, cobalt-chromium, cobalt-chromium alloys, stainless steel, PEEK, bone, polymers, or other suitable metal or non-metal material and combinations and composites thereof. Of course, it is understood that the relative size of the components can be modified for the particular vertebra(e) to be instrumented and for the particular location or structure of the vertebrae to which the anchor assembly will be engaged.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. An implant for insertion in a disc space between endplates of adjacent vertebrae, comprising:
a first elongated spacer member extending between a leading insertion end and an opposite trailing end of the implant, said first spacer member including an inferior bone engaging surface and an opposite superior bone engaging surface that each extend between said leading and trailing ends and further extend between opposite side walls of said spacer member, wherein said superior and inferior bone engaging surfaces are convexly curved at least in a direction between said leading end and said trailing end;
a second elongated spacer member extending between said leading insertion end and said opposite trailing end of the implant, said second spacer member including a second inferior bone engaging surface and an opposite second superior bone engaging surface that each extend between said leading and trailing ends and further extend between opposite side walls of said second spacer member, wherein said second superior and inferior bone engaging surfaces of said second spacer member are convexly curved at least in a direction between said leading end and said trailing end; and
a housing extending between and receiving said first and second spacer members to maintain said first and second spacer members in spaced relation to one another, wherein each of said first and second spacer members is rotatable in said housing from a first orientation wherein said sidewalls of each first and second spacer members are positioned to face a respective one of the endplates of the adjacent vertebrae to a second orientation wherein each of said inferior and superior bone engaging surfaces of said first and second spacer members face the respective endplate of the adjacent vertebrae, and in said first orientation said first and second spacer members each define a maximum height between said side walls thereof that is less than a maximum implanted height between said inferior and superior bone engaging surfaces of each of said first and second spacer members.

2. The implant claim 1, wherein said side walls of said first spacer member are parallel to one another and said side walls of said second spacer member are parallel to one another, and when said first and second spacer members are in said second orientation said side walls of said first spacer member are parallel to said sidewalls of said second spacer member.

3. The implant of claim 1, wherein:
said superior and inferior bone engaging surfaces of said first and second spacer members include bone engagement features selected from the group consisting of grooves, recesses, ridges, serrations, knurlings, spikes, or roughened surfaces; and
first and second spacer members each include at least one cavity extending between and opening at each of said superior and inferior bone engaging surfaces thereof.

4. The implant of claim 1, wherein said first and second spacer members each include a length extending from said leading end to said trailing end of the implant, and said housing is located about mid-length of said first and second spacer members.

5. The implant of claim 4, wherein:
said first spacer member includes a first portion extending from said housing to said leading end of the implant and a second portion extending from said housing to said trailing end of the implant, wherein each of said first and second portions of said first spacer member includes a part of said superior and inferior bone engaging surfaces of said first spacer member and each of said parts of said bone engaging surfaces is convexly curved from said housing to said respective leading or trailing end of the implant; and
said second spacer member includes a first portion extending from said housing to said leading end of the implant and a second portion extending from said housing to said trailing end of the implant, wherein each of said first and second portions of said second spacer member includes a part of said superior and inferior bone engaging surfaces of said second spacer member and each of said parts of said bone engaging surfaces is convexly curved from said housing to said respective leading or trailing end of the implant.

6. The implant of claim 5, wherein:
said first and second portions of each of said first and second spacer members are connected to one another with a neck that extends through said housing;
when said first and second spacer members are in said second orientation:
inner side walls of the implant are formed by one of said side walls of said first spacer member facing one of said side walls of said second spacer member and outer side walls of the implant are formed by other of said side walls of said first and second spacer members; and
said outer sidewall formed by said first spacer member defines a maximum height between said leading and trailing ends of the implant that is less than a maximum height of each of said inner side walls of said first and second spacer members between said leading and trailing ends thereof, and said maximum height of each of said inner side walls is less than a maximum height of said outer side wall formed by said second spacer member between said leading end and said trailing end.

7. The implant of claim 6, wherein said superior and inferior bone engaging surfaces each define a convexly curved profile that extends from one of said outer side walls to the other of said outer side walls.

8. The implant of claim 1, wherein each of said first and second spacer members includes a nose at said leading end of the implant that is convexly curved from said inferior bone engaging surface to said superior bone engaging surface.

9. The implant of claim 1, wherein said housing extends between and rotatably receives each of said first and second spacer members at said leading end of the implant.

10. The implant of claim 1, further comprising a cap extending between and connected to each of said trailing ends of said first and second spacer members.

11. The implant of claim 10, wherein said superior and inferior bone engaging surfaces of each of said first and second spacer members includes a groove extending therein and said cap includes a four projecting members positioned in respective ones of said grooves when said cap is connected to each of said trailing ends of said first and second spacer members, and further comprising a pair of fasteners extending through said cap and engaging said cap to said trailing ends of said first and second spacer members.

12. An implant for insertion in a spinal disc space between endplates of adjacent vertebrae, comprising:
a central housing defining at least one receptacle opening at opposite sides of said housing;
a first elongate body positioned in said receptacle of said housing and extending outwardly from said opposite sides of said housing; and
a second elongate body positioned in said receptacle of said housing and extending outwardly from said opposite sides of said housing in side-by-side relation to said first elongate body, said first and second elongate bodies each defining a length extending from a leading end of the implant on one side of said housing to an opposite trailing end of the implant on an opposite side of said housing, each of said first and second elongate bodies further defining a maximum height between oppositely facing superior and inferior bone engaging surfaces thereof and a width between oppositely facing outer side walls thereof, wherein said length is substantially greater than said maximum height, and said maximum height is greater than said width.

13. The implant of claim 12, wherein:
when said first and second elongate bodies are implanted in the disc space said lengths of said first and second elongate bodies extend transversely to the sagittal plane so that said first and second elongate bodies each extend from a first lateral edge of the adjacent vertebrae to an opposite lateral edge of the adjacent vertebrae;
said first elongate body is positioned posteriorly of said second elongate body in said housing so that when said first and second elongate bodies are implanted in the disc space said second elongate body extends across an anterior portion of the disc space and said first elongate body extends along a central portion of the disc space; and
said length of said first elongate body is greater than said length of said second elongate body.

14. The implant of claim 12, wherein:
said superior and inferior bone engaging surfaces of each of said first and second elongate bodies are convexly curved in a direction extending from said leading end toward said trailing end, and said superior and inferior surfaces of each of said first and second elongate bodies are convexly curved in a direction extending between said opposite side walls thereof; and
said side walls of said first elongate body are parallel to one another and said side walls of said second elongate body are parallel to one another.

15. The implant of claim 14, wherein:
said first and second elongate bodies each extend along a central longitudinal axis and each of said first and second elongate bodies are rotatable around said central longitudinal axis thereof in said housing from a first orientation for insertion of the implant into the disc space where said side walls of each of said first and second elongate bodies face respective ones of the endplates of the adjacent vertebrae to a second orientation where said superior and inferior bone engaging surfaces of each of said first and second elongate bodies face respective ones of the endplates of the adjacent vertebrae.

16. The implant of claim 12, wherein:
said first elongate body includes a first elongate portion extending from said housing to said leading end and a second elongate portion extending from said housing to said trailing end;
said second elongate body includes a first elongate portion extending from said housing to said leading end and a second elongate portion extending from said housing to said trailing end;
said first and second elongate portions of said first elongate body have substantially the same length extending from said housing to respective ones of said leading and trailing ends; and
said first and second elongate portions of said second elongate body have substantially the same length extending from said housing to respective ones of said leading and trailing ends.

17. The implant of claim 16, wherein:
said central housing defines an elongated body having a first node defining a first receptacle and a second node beside said first node, said second node defining a second receptacle;
said first elongate body includes a neck extending through said first receptacle that connects said first and second elongate portions thereof;
said second elongate body includes a neck extending through said second receptacle that connects said first and second elongate portions thereof; and
said necks of said first and second elongate bodies are each rotatable in said housing to rotate said first and second elongate bodies about a central longitudinal axis thereof.

18. An implant for insertion in a spinal disc space between endplates of adjacent vertebrae, comprising:
a housing defining at least one receptacle opening at opposite sides of said housing, said receptacle further being elongate between opposite ends of said housing; and
a first elongate body positioned in said receptacle of said housing and extending outwardly from at least one of said opposite sides of said housing along a first central longitudinal axis, wherein said first elongate body is rotatable relative to said housing around said first central longitudinal axis between a reduced height orientation for insertion in the disc space and a maximum height orientation for implantation in the disc space, said reduced height orientation being sized to space said first elongate body from at least one of the endplates of the vertebrae when said first elongate body is implanted in the disc space in its reduced height orientation and said maximum height orientation being sized so that first elongate body contacts each of the endplates of the vertebrae when said first elongate body is rotated for implantation in the disc space.

19. The implant of claim 18, wherein said first elongate body extends outwardly from each of said opposite sides of said housing and further comprising:
a second elongate body positioned in said receptacle of said housing and extending outwardly from said opposite sides of said housing along a second central longitudinal axis, wherein said second elongate body is rotatable relative to said housing and rotatable relative to said first elongate body around said second central longitudinal axis between a reduced height orientation for insertion in the disc space and a maximum height orientation for implantation in the disc space, said reduced height orientation being sized to space said second elongate body from at least one of the endplates of the adjacent vertebrae when said second elongate body is positioned in the disc space in its reduced height orientation and said maximum height orientation being sized so that said second elongate body contacts each of the endplates of the vertebrae when said second elongate body is rotated for implantation in the disc space.

20. The implant of claim 18, wherein:
said housing includes a lipped region along at least one side thereof that extends along said receptacle and said housing further includes gear teeth extending along said lipped region between said opposite ends of said receptacle; and
said elongate body includes a neck extending from a leading end of said elongate body and said neck is positioned in said receptacle of said housing, said elongate body further comprising a head at an end of said neck, said head including a plurality of teeth extending around a perimeter of said head, said teeth of said head meshing with said gear teeth of said housing to facilitate rotation of said elongate body from said reduced height orientation to said maximum height orientation and to move said elongate body toward one of said opposite ends of said receptacle of said housing.
